(19) United States
(12) Patent Application Publication
(10) Pub. No.: US 2026/0014004 A1
(43) Pub. Date: Jan. 15, 2026

(54) DEVICES AND METHODS FOR DELIVERING NON-BALLOON EXPANDABLE IMPLANTS

(71) Applicant: Starlight Cardiovascular, Inc., San Diego, CA (US)

(72) Inventors: Beverly T. Tang, La Jolla, CA (US); Kathryn A. Olson, San Diego, CA (US); Mark Juravic, Encinitas, CA (US); Jerin Brust, Del Mar, CA (US); Christian G. Monroe, Fishkill, NY (US)

(21) Appl. No.: 19/333,908

(22) Filed: Sep. 19, 2025

Related U.S. Application Data
(63) Continuation of application No. PCT/US24/20720, filed on Mar. 20, 2024.
(60) Provisional application No. 63/491,128, filed on Mar. 20, 2023.

(51) Int. Cl. A61F 2/82; A61F 2/95

(52) U.S. Cl. CPC A61F 2/82; A61F 2/95; A61F 2250/0058

(57) ABSTRACT

The devices and methods described herein are for use with conventional catheters and guidewires. Access can be maintained with the catheter and/or guidewire and the device can be backloaded onto the guidewire and into the catheter to be advanced to a target. The devices can include a tubular shaft having a lumen for receiving a guidewire; at least one aperture for receiving a feature of an implant positioned on the tubular shaft; and a plurality of relief cuts. The outer diameter of the tubular shaft is sized relative to an inner diameter of a catheter for receiving the tubular shaft, such that a sheath is not positionable in a lumen of the catheter when the tubular shaft is in the lumen of the catheter. The tubular shaft can be axially displaced in the lumen of the catheter to advance or retract the implant positioned on the tubular shaft.

### CROSS-REFERENCE TO RELATED APPLICATIONS

This application is a continuation of PCT Application Ser. No. PCT/US2024/020720, filed Mar. 20, 2024, which claims the priority benefit of U.S. Provisional Patent Application Ser. No. 63/491,128, filed Mar. 20, 2023, the contents of which is herein incorporated by reference in its entirety.

### TECHNICAL FIELD

This disclosure relates generally to the field of interventional medicine, and more specifically to the field of intravascular stenting. Described herein are stent delivery devices and methods.

### BACKGROUND

Self-expanding stents are used in a wide variety of clinical application, including interventions for coronary artery diseases, congenital heart diseases, gastrointestinal tract disorders, peripheral vessel diseases, and the like. Conventional systems to deliver self-expanding stents include “over the wire” systems that include stents that are crimped onto a tube and contained within a sheath, and the whole system is pushed into or through a delivery catheter and/or over a wire. These systems can be very stiff. Alternatively, conventional systems to delivery self-expanding stents can include “empty catheter” systems that have stents that are crimped onto a pusher wire and contained within a sheath. The stent is then transferred out of the sheath and into the back of the delivery catheter using the pusher wire and then pushed up the length of the delivery catheter in its unsheathed form.

### SUMMARY

In some aspects, the techniques described herein relate to a system for delivering a vascular implant, the system including: a tubular shaft defining a lumen and including: a proximal section, an implant coupling section bonded to the proximal section, wherein the implant coupling section defines at least one aperture, and wherein the at least one aperture is configured to receive a feature of an implant positioned on an outer diameter of the tubular shaft, and a plurality of relief cuts in the tubular shaft to increase a flexibility of the tubular shaft.

In some aspects, the techniques described herein relate to a method for delivering a vascular implant, the method including: advancing a guidewire and a catheter to a target site, wherein the guidewire is configured to be axially translated in the catheter; loading a tubular shaft over the guidewire and into a lumen of the catheter, wherein at least a portion of the tubular shaft has an implant disposed thereon; and axially translating the tubular shaft over the guidewire and through the lumen of the catheter.

In some aspects, the techniques described herein relate to a system for delivering a vascular implant, the system including: a tubular shaft defining a lumen and including: a proximal section, an implant coupling section defining at least one aperture, and a plurality of relief cuts in the tubular shaft; an implant having a proximal end including at least one feature, wherein the implant is positioned on the tubular shaft, and the at least one feature is positioned in the at least one aperture of the tubular shaft; and a catheter defining a lumen having an inner diameter, wherein the inner diameter of the catheter is sized relative to an outer diameter of the tubular shaft such that a sheath is not positionable in the lumen of the catheter when the tubular shaft is in the lumen of the catheter, wherein the tubular shaft is configured to be axially displaced in the lumen of the catheter to advance or retract the implant positioned on the tubular shaft.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 shows a catheter over a guidewire in a target vessel.

FIG. 2A shows a distal portion of an embodiment of a self-expanding stent crimped onto a tubular shaft, positioned over a guidewire and within a lumen of a catheter.

FIG. 2B shows a cross-sectional schematic of a guidewire within a tubular shaft, which is within a catheter.

FIG. 2C shows a cross-sectional schematic of a guide within a tubular shaft, which is within a catheter, and the tubular shaft has an implant positioned thereon.

FIG. 3 shows a proximal portion of an embodiment of a self-expanding stent crimped onto a tubular shaft, positioned over a guidewire and within a lumen of a catheter.

FIG. 4 shows a zoomed-in view of a distal portion of an embodiment of a stent crimped onto a tubular shaft.

FIG. 5 shows a zoomed-in view of a proximal portion of an embodiment of a stent crimped onto a tubular shaft.

FIG. 6 shows a zoomed-in view of an aperture defined by a proximal portion of the tubular shaft, the aperture being sized and shaped to receive a radiopaque maker on a proximal portion of the stent therein.

FIG. 7A shows a schematic of an embodiment of a tubular shaft.

FIG. 7B shows a schematic of a portion of a tubular shaft.

FIG. 8 shows an embodiment of a proximal section of a tubular shaft.

FIG. 9 shows an embodiment of a distal section of a tubular shaft.

FIG. 10A shows a perspective view of an embodiment of an implant coupling section of a tubular shaft.

FIG. 10B shows a perspective view of an embodiment of an implant coupling section of a tubular shaft.

FIG. 11 shows a side view of the implant coupling section of FIG. 10B.

FIG. 12 shows a perspective view of an embodiment of an implant coupling section of a tubular shaft.

FIG. 13 shows a magnified view of a portion of a tubular shaft having an implant radiopaque marker coupled thereto.

FIG. 14 shows a magnified view of a portion of a tubular shaft having an implant coupled thereto.

FIG. 15 shows a two-dimensional crimped view of an embodiment of an implant.

FIG. 16 shows a zoomed in view of a radiopaque marker of the implant of FIG. 15.

FIG. 17 is a schematic of an embodiment of a method of delivering an implant to a target site.

FIG. 18 is a schematic of an embodiment of a method of delivering an implant to a target site.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems, devices, and methods described herein may be used for coronary applications, peripheral applications, neurovascular applications, and/or pediatric applications. For example, the systems, devices, and methods described herein may provide an improved way of navigating implants through or to vessels that experience: substantial changes between a systolic diameter and a diastolic diameter, forces from flow patterns that may cause an implant to migrate, changes in anatomy (e.g., a ductus arteriosus attempting to close), or other situations causing substantial forces or diameter changes.

In some embodiments, the systems, devices, and methods described herein may be used for treating and/or managing patient conditions associated with a ductus arteriosus or other tortuous anatomy. These embodiments may provide technical solutions to address the challenges facing treating physicians including a delivery system particularly sized for the target population (e.g., pediatrics); insertion, navigation, and deployment through tortuous anatomy; and precise implant placement, which can also ensure avoidance of additional surgeries to adjust and correct the placement of the implant.

In general, an implant as used herein may refer to a stent, a self-expanding stent, a stentriever, a flow diverter, a clot capture device, a septal conduit, flow occlude, flow restrictor, and the like.

More specifically, the technical problems with conventional delivery systems for self-expanding stent applications are multi-fold. Conventional delivery systems for self-expanding stents are cumbersome and typically include the use of multiple shafts including at least a pusher wire having the stent positioned thereon and a sheath positioned over the stent, all of which is positioned within a delivery catheter. The delivery systems described herein provide a technical solution to the above technical problems. For example, the delivery systems described herein provide fewer components than conventional systems to simplify and/or streamline setup and stent delivery. In addition, the delivery systems described herein include a tubular shaft onto which the stent is positioned or crimped. A proximal portion of the tubular shaft includes one or more keyed surfaces or cutouts (also known as, apertures or windows) that are shaped to receive one or more features (e.g., radiopaque markers) of the stent. As such, the tubular shaft also functions to advance and retract the stent in the catheter lumen before stent deployment.

A further technical problem of conventional delivery systems generally is that they limit a physician's ability to select an appropriate or desired catheter type and/or size, since such selections are typically limited to the specifics of the delivery system. The delivery systems described herein provide a technical solution to this technical problem By providing a tubular shaft with the stent positioned or crimped thereon, which can be delivered over a wire within a catheter that is already parked or disposed at a target site (i.e., access is maintained) may enable the physician to select a desired catheter type and/or size, which ensures that the physician is not limited to a particular catheter packaged with (or otherwise assigned to) the delivery system. Accordingly, the stent delivery systems and methods described herein reduce the number of components of the system and enable a physician to select a catheter size that is appropriate for the patient and/or target anatomy.

Further, the technical solutions provided by the systems, devices, and methods described herein provide for any one or more of: accessing a target anatomy with any catheter and/or any guidewire; maintaining guidewire position (e.g., based on procedure, physician, etc.), so access is not lost; backloading an implant over the guidewire with a tubular shaft; positioning the tubular shaft and implant into an empty catheter (e.g., no sheath constraining the implant within the catheter) that is already in place (i.e., no need to exchange an existing catheter for a different delivery catheter); maintaining both the guidewire and the catheter position across or within a target vessel; an option to exchange an existing guidewire to a different guidewire using the systems described herein without removing the tubular shaft and/or implant from the catheter; and/or adjusting the amount of leading guidewire and location of guidewire access.

The technical solutions provided by the various embodiments of tubular shafts described herein may include any one or more of: apertures or other features on a proximal and/or distal end portion of the tubular shaft to allow engagement with the implant; apertures or other features on a proximal and/or distal end portion of the tubular shaft to allow the implant to be removed from the delivery system (at the proximal or operator end of the system); apertures or other features on a proximal and/or distal end portion of the tubular shaft to provide redundancy in the engagement mechanism and still allow for pushing (advancing) and pulling (retracting) of the implant even when an end or a feature or aperture becomes disengaged; maximization of an inner lumen defined by the tubular shaft for a guidewire because there is no sheath enclosing the implant, to save wall thickness; the ability to use any catheter of an appropriate inner diameter and still have guidewire access for implant deployment; implant stability and support provided while still maintaining flexibility for delivery through tortuous vessels; and an ability to match a flexibility of the implant with a flexibility of the tubular shaft using cut patterns and/or materials.

Described herein are delivery systems that include non-balloon-expandable stents that can be positioned or crimped onto a tubular shaft. The tubular shaft may include features that are used to engage and push (e.g., advance) or pull (e.g., retract) the stent in a lumen of a catheter.

Alternatively, the implant may not be crimped onto the tubular shaft, but may instead be coupled to a distal end portion of the tubular shaft (using one or more features of the tubular shaft that are shaped and/or positioned to interact with the implant) and axially translated in the lumen of the catheter. For example, as will be described in further detail elsewhere herein, a tubular shaft may include a proximal section and implant coupling section, without having a distal section.

In general, the methods and devices described herein may be used in any application in which a non-balloon-expandable stent or implant is navigated in the vasculature and/or delivered to a target site. In some embodiments, guidewire access may be maintained and separated from delivery, such that the devices and methods described herein can be used with a guidewire positioned in the vasculature before introduction of delivery system components. The systems described herein can be considered “over-the-wire” such that guidewire access can be maintained during a procedure. The tubular shaft defines a lumen for receiving a guidewire therethrough. Said another way, the tubular shaft may maintain or define a guidewire lumen, such that the tubular shaft can be backloaded over the guidewire.

The tubular shaft described herein may include or be formed of a laser-cut metallic shaft. For example, the tubular shaft can be made of or formed of a laser cut metal hypotube (e.g., Nitinol, stainless-steel, etc.) or a polymer or shafts that are joined to each other to create the desired flexibility profile (i.e., a laser cut Nitinol distal tip for flexibility joined to a polymer or stainless-steel end for push force transmission). For example, when the tubular shaft includes a material, like Nitinol, there may be fewer cuts in the cut pattern because the material is more flexible. For example, when the tubular shaft includes a material, like Stainless steel, there may be more cuts in the cut pattern because the material is less flexible. The laser-cut tubular shafts described herein are designed for flexibility and translation of push and pull forces. For example, the tubular shaft may include offset cuts so that the tube does not forelengthen or foreshorten.

The systems and devices described herein may function to deliver an implant to a target site in a vessel over the wire using an empty catheter (e.g., no sheath constraining the implant in the catheter). Such systems and devices can be used for intravascular interventions, for example coronary, neurovascular, peripheral, arterial, and/or venous, etc. in adults and pediatrics.

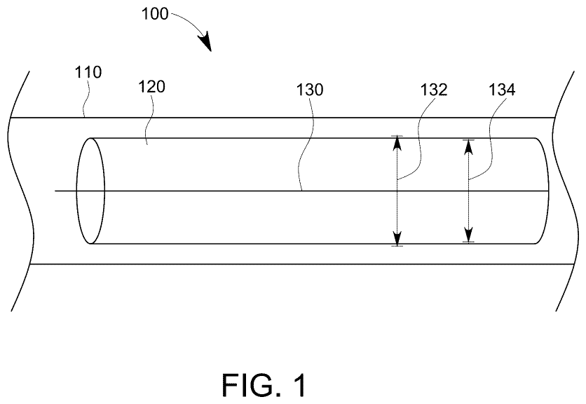

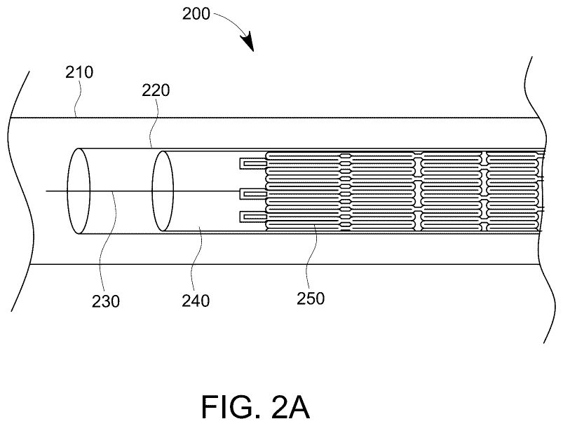

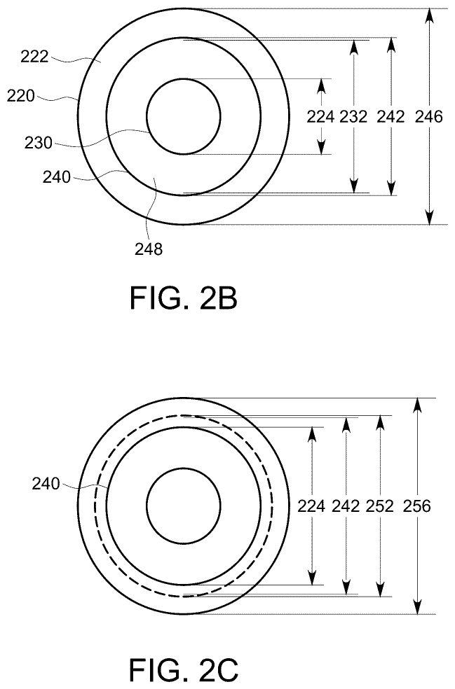

FIG. 1 shows a distal portion 100 of a catheter 120 over a guidewire 130 in a target vessel 110. The guidewire 130 is advanced through the vessel 110 to a target site or anatomy, and the catheter 120 is advanced over the guidewire 130 to the target site or anatomy. The catheter 120 has an outer diameter 132 and an inner diameter 134 (also shown in FIGS. 2B-2C). FIG. 1 represents access to a target anatomy or target site in a vessel 110. The target anatomy or target site may be a ductus arteriosus. The target anatomy or target site may be a coronary artery. The target anatomy or target site may be an intracranial site. The target anatomy or target site may be vasculature (venous or arterial) in the periphery. The target anatomy or target site may be a pulmonary artery fluidly connected to a ductus arteriosus. The target anatomy or target site may be an aorta fluidly connected to a ductus arteriosus.

FIGS. 2A-2C illustrate sideview and cross-sectional views, respectively, of a distal portion 200 of a delivery system. For example, as shown in FIG. 2A, the delivery system includes implant 250 (e.g., stent) positioned over at least a portion of a tubular shaft 240. The tubular shaft 240 is positionable over a guidewire 230 within a lumen 222 of a catheter 220. For example, the tubular shaft 240 and implant 250 may be backloaded into a lumen 222 of the catheter 220 over the guidewire 230 and advanced through the lumen 222 of the catheter 220 to the target anatomy in the vessel 210. As shown in FIG. 2B, an outer diameter 242 of at least a portion of the tubular shaft 240 (e.g., a distal section of the tubular shaft) is less than an inner diameter 224 of the catheter 220 and the tubular shaft 240 is shown as positioned in a lumen 222 of the catheter 220. In some embodiments, the outer diameter 242 of at least a portion of the tubular shaft 240 (e.g., a proximal section) may be about 85% to about 98% of the inner diameter 224 of the catheter 220; about 85% to about 90% of the inner diameter 224 of the catheter 220; about 95% to about 98% of an inner diameter 224 of the catheter 220; about 88% to about 95% of an inner diameter 224 of the catheter 220; or about 90% to about 97% of an inner diameter 224 of the catheter 220. Further, for example, the outer diameter 242 of at least a portion of the tubular shaft 240 (e.g., a distal section) may be about 65% to about 95% of the inner diameter 224 of the catheter 220; about 70% to about 80% of the inner diameter 224 of the catheter 220; about 80% to about 90% of an inner diameter 224 of the catheter 220; about 70% to about 75% of an inner diameter 224 of the catheter 220; about 85% to about 90% of an inner diameter 224 of the catheter 220; or about 80% to about 83% of an inner diameter 224 of the catheter 220.

As shown in FIG. 2B, an outer diameter 232 of a guidewire 230 is less than an inner diameter 246 of at least a portion of a tubular shaft 240. In this example, the guidewire 230 is shown as positioned in a lumen 248 of at least a portion of the tubular shaft 240. For example, an outer diameter 323 of a guidewire 230 may be about 75% to about 85% of an inner diameter 246 of at least a portion of a tubular shaft 240 (e.g., a proximal section); about 75% to about 80% of the inner diameter 246 of at least a portion of the tubular shaft 240; about 76% to about 81% of the inner diameter 246 of at least a portion of the tubular shaft 240; about 77% to about 80% of the inner diameter 246 of at least a portion of the tubular shaft 240.

Further, for example, an outer diameter 323 of the guidewire 230 may be about 85% to about 95% of the inner diameter 246 of at least a portion of a tubular shaft 240 (e.g., a distal section); about 88% to about 93% of the inner diameter 246 of at least a portion of the tubular shaft 240; about 89% to about 91% of the inner diameter 246 of at least a portion of the tubular shaft 240; or about 90% to about 94% of the inner diameter 246 of at least a portion of the tubular shaft 240.

As shown in FIG. 2C, an outer diameter 242 of at least a portion of the tubular shaft 240 (e.g., a distal section of the tubular shaft, when present) is less than an inner diameter 252 of a crimped implant 250 or an implant 250 positioned on or coupled to the tubular shaft. For example, the diameter 242 of at least a portion of the tubular shaft 240 may be about 75% to about 98% of the inner diameter 252 of the crimped implant positioned on the tubular shaft. The diameter 242 of at least a portion of the tubular shaft 240 may be about 75% to about 85% of the inner diameter 252 of the crimped implant positioned on the tubular shaft. The diameter 242 of at least a portion of the tubular shaft 240 may be about 90% to about 98% of the inner diameter 252 of the crimped implant positioned on the tubular shaft. The diameter 242 of at least a portion of the tubular shaft 240 may be about 85% to about 97% of the inner diameter 252 of the crimped implant positioned on the tubular shaft. Further, an outer diameter 256 of an implant 250 positioned about the tubular shaft 240 is less than an inner diameter 224 of the catheter 220. For example, an outer diameter 256 of the implant 250 may be about 90% to about 99% of the inner diameter 224 of the catheter 220. The outer diameter 256 of the implant 250 may be about 90% to about 95% of the inner diameter 224 of the catheter 220. The outer diameter 256 of the implant 250 may be about 93% to about 98% of the inner diameter 224 of the catheter 220.

In some embodiments, an inner diameter of the tubular shaft can be lined with or include a lubricious material (e.g., silicone, PTFE, etc.) coating to reduce friction during guidewire access or guidewire translation in the lumen of the tubular shaft.

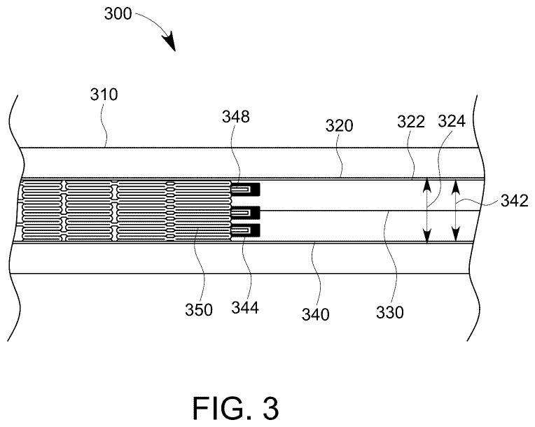

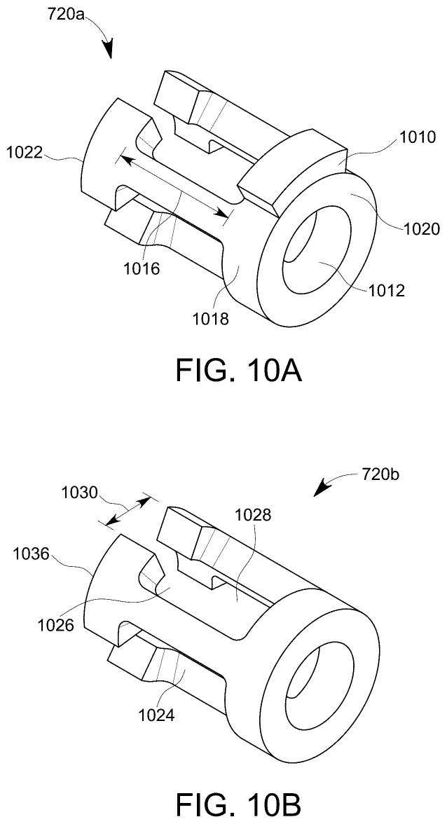

FIG. 3 shows a proximal portion 300 of a delivery system. For example, the delivery system includes an implant 350 positioned over a tubular shaft 340. The tubular shaft 340 is positionable over a guidewire 330 within a lumen of a catheter 320. The proximal portion of the tubular shaft 340 defines one or more apertures or windows 344, examples of which will be described with respect to FIGS. 10A-13 below. The one or more apertures or windows 344 are sized and/or shaped to receive one or more features 349 (e.g., radiopaque markers) of the implant 350 therein. Although one or more apertures or windows 344 are shown in a proximal portion or section of an outer diameter of the tubular shaft 340, one or more apertures or windows 344 may additionally, or alternatively, be in a distal portion of the tubular shaft 340 (e.g., distal section) or in an intermediate portion (e.g., implant coupling section) of the tubular shaft 340. For example, an implant 350 that interacts with the tubular shaft 340 may include one or more radiopaque markers 348 on a proximal end and/or distal end, such that the radiopaque markers 348 interact with the one or more apertures or windows 344 to enable the implant 350 to be pushed or pulled relative to the catheter 320 and/or guidewire 330.

Alternatively, one or more other features of the implant may engage with one or more elements of the tubular shaft to advance and retract the stent. For example, the one or more elements of the tubular shaft may have a larger outer diameter than the crimped stent (or another portion of the tubular shaft) to push the stent. Further, for example, the one or more elements of the tubular shaft may have a smaller outer diameter than the crimped stent, such that the one or more elements fit under the stent and engage with the stent to push or pull the stent through the lumen of the catheter.

In some embodiments, the implant may be translated with the tubular shaft without engagement of the implant radiopaque markers or other features. This may occur when an outer diameter of at least a portion of the tubular shaft substantially matches or is substantially similar to an outer diameter of the implant when in a lumen of the catheter. This allows for the implant to be pushed down a catheter, in the distal direction, however may not allow for the implant to be pulled back in the proximal direction within the catheter.

In some embodiments, the implant may engage with a proximal and/or distal portion of the tubular shaft using one or more locking features (e.g., a complementary female and male connector) between the implant and the tubular shaft.

Further, as shown in FIG. 3 and described above with reference to FIGS. 2B-2C, the catheter 320 defines a lumen 322 having an inner diameter 324 to receive the tubular shaft 340 therein. The inner diameter 324 of the catheter 320 is sized relative to the outer diameter 342 of the tubular shaft 340 such that a sheath is not positionable in a lumen 322 of the catheter 320 when the tubular shaft 340 is in the lumen 322 of the catheter 320.

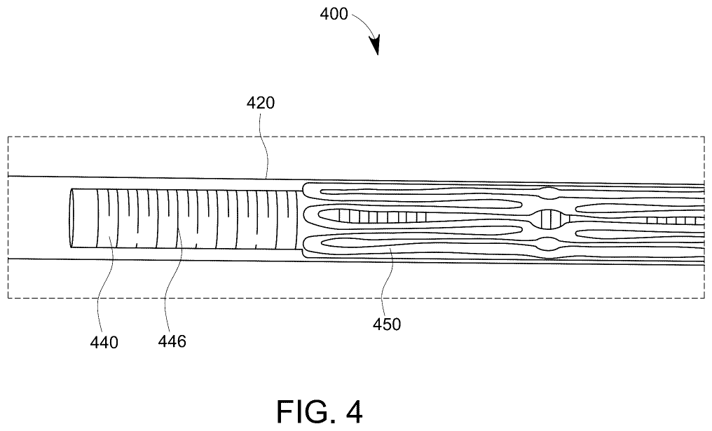

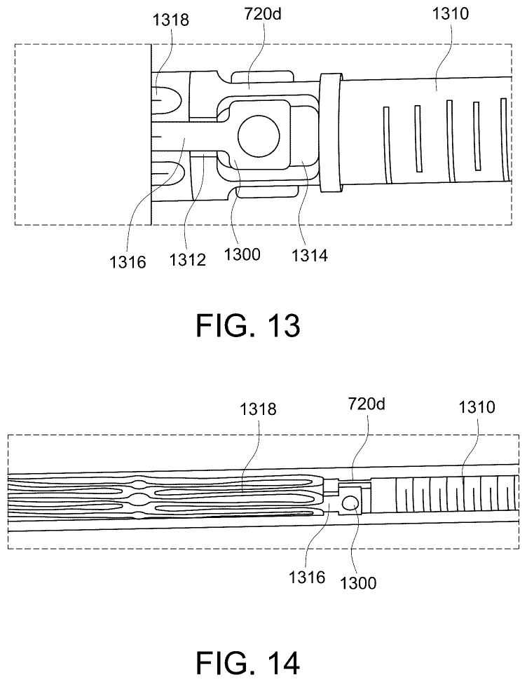

FIG. 4 shows a zoomed-in view of a distal portion 400 of an embodiment of an implant 450 crimped onto (or positioned on) a tubular shaft 440. The implant 450 positioned on the tubular shaft 440 is shown positioned in a lumen of a catheter 420. The tubular shaft 440 includes one or more relief cuts 446 (e.g., a cut pattern, optionally laser cut) for flexibility of the tubular shaft 440. In some embodiments, one or more radiopaque markers (shown and described with reference to FIGS. 13-16) on a distal portion of the implant interact with one or more apertures or windows in a portion of an outer diameter of the tubular shaft. For example, the one or more windows or apertures in the tubular shaft can engage radiopaque markers on both sides of the implant (i.e., distal and proximal sides of the stent) or on one side (i.e., proximal side). Various apertures or windows will be described with reference to FIGS. 10A-12.

In some configurations of the systems described herein, the tubular shaft may include a backstop 1010, a bumper, ring, raised shaft, or the like, as shown in FIG. 10A, on an outer diameter of the tubular shaft on a proximal end of the one or more windows or apertures 1016 to push against the radiopaque markers (or other features) of the implant to advance the implant through a lumen of a catheter.

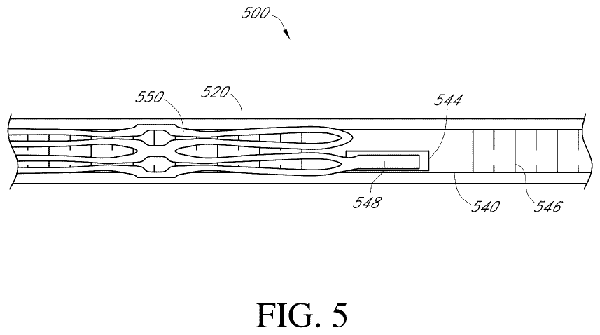

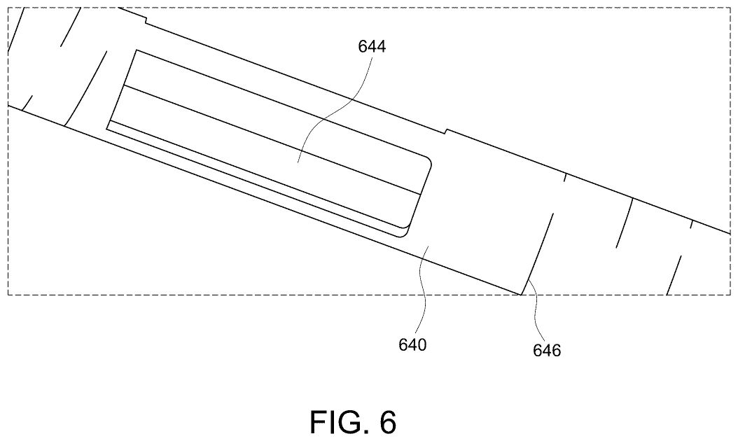

FIG. 5 shows a zoomed-in view of a proximal portion 500 of an embodiment of an implant 550 crimped onto or positioned on a tubular shaft 540. As shown in FIG. 5, one or more radiopaque markers 548 (or other features) of the implant 550 are engaged with one or more windows or apertures 544 of the tubular shaft 540. Further as shown, the implant 550 positioned on the tubular shaft 540 is positioned in a catheter 520 and is translated axially (or axially displaced) within an inner diameter of the catheter 520. For example, the implant 550 is axially translated in a lumen of the catheter 520 at least in part through interaction of the one or more radiopaque markers 548 (or other features) of the implant 550 with the one or more windows or apertures 544 of the tubular shaft 540. The tubular shaft 540 includes one or more relief cuts 546 for flexibility of the tubular shaft 540.

FIG. 6 shows a zoomed-in view of an aperture 644 defined by a portion of the tubular shaft 640. The aperture 644 is sized and shaped to receive a radiopaque maker (or other feature) of the implant therein or couple to the radiopaque marker (or other feature), so that the aperture 644 can be used to advance or retract the implant relative to the catheter. FIG. 6 also shows a zoomed-in view of the relief cuts 546 of the tubular shaft 640. The relief cuts 546 are arranged in an interrupted spiral cut pattern but could alternatively be arranged in a brick cut pattern. For example, the tubular shaft 640 may include a spine (e.g., axial or spiral) to enable both pushing and pulling without causing compression or elongation of the tubular shaft 640.

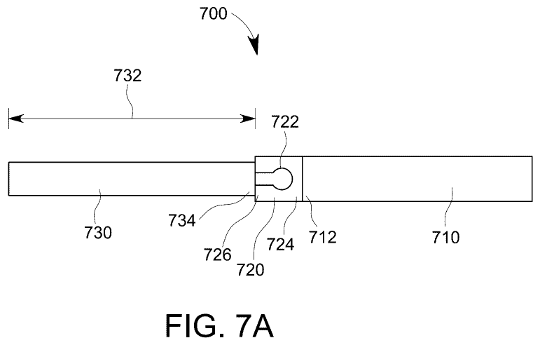

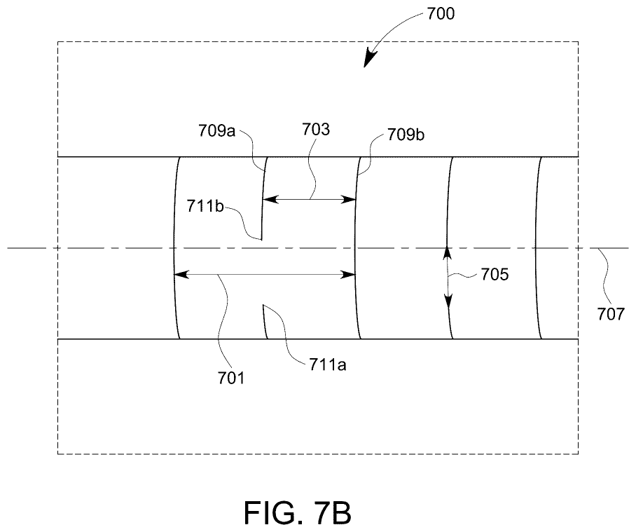

FIG. 7A shows an embodiment of a tubular shaft 700. The tubular shaft 700 includes a proximal section 710, an implant coupling section 720, and a distal section 730. In some embodiments, the tubular shaft 700 includes the proximal section 710 and the implant coupling section 720, but does not include the distal section 730. The proximal section 710 functions for pushability of the tubular shaft. The implant coupling section 720 (see, for example FIGS. 10A-12) functions to reversibly couple to an implant, for example using one or more complementarily shaped windows or apertures 722 that couple to a radiopaque marker or other feature of the implant. The distal section 730 functions for navigating an implant through the vasculature and over a guidewire. The distal section 730 includes an implant receiving section 732. For example, at least a portion of an implant may overlay the implant receiving section 732. The distal section 730 may have less deflection than the implant to increase flexibility of the combination of the distal section 730 and the implant positioned thereon to increase deliverability.

A distal end 712 of the proximal section 710 may be bonded to a proximal end 724 of the implant coupling section 720. In some embodiments, the proximal section 710 and the implant coupling section 720 are a monolithic shaft or a continuous tube.

A distal end 726 of the implant coupling section 720 may be bonded to a proximal end 734 of the distal section 730. The bonding between adjacent sections may be formed by welding, soldering, brazing, and the like. In some embodiments, bonding between the proximal section 710 and the implant coupling section 720 may use a first form of bonding (e.g., welding, soldering, brazing, etc.), and bonding between the implant coupling section 720 and the distal section 730 may use another form of bonding, different from the first bonding form. In some embodiments, bonding between the proximal section 710 and the implant coupling section 720 may use a first material, and bonding between the implant coupling section 720 and the distal section 730 may use another material, different from the first material. In some embodiments, the bond between the proximal section 710 and the implant coupling section 720 and the bond between the implant coupling section 720 and the distal section 730 includes the same material and/or uses the same process (e.g., welding, soldering, brazing, etc.).

The proximal section 710 may have an outer diameter that is greater than an outer diameter of the distal section 730. For example, an outer diameter of the proximal section 710 may be about 110% to about 135% greater than an outer diameter of the distal section 730; about 115% to about 130% greater than an outer diameter of the distal section 730; about 115% to about 120% greater than an outer diameter of the distal section 730; or about 125% to about 130% greater than an outer diameter of the distal section 730. The implant coupling section 720 may have a substantially similar outer diameter as the proximal section 710. The change in outer diameter from the proximal section 710 or implant coupling section 720 to the outer diameter of the distal section 730 may be a step down (i.e., not a tapered or gradual transition). In some embodiments, the transition between the proximal section 710 or the implant coupling section 720 and the distal section 730 is gradual or tapered, based on a tapering, or grading, of each section and/or the bonding process. For example, the bonding process may be such that the change in outer diameter is tapered from the greater outer diameter to the lesser outer diameter through tapering of the bonding material.

In some embodiments, a tubular shaft has a monolithic construction, for example a laser cut hypotube. In such embodiments, the outer diameter of the tubular shaft may be substantially uniform.

In some embodiments, the proximal section 710, implant coupling section 720, and distal section 730 are each formed of, comprise, or include, for example, stainless steel, a titanium alloy, platinum, tantalum, palladium, a combination thereof, or the like. For example, in some embodiments, each section is formed of stainless steel.

FIG. 7B shows a schematic of a portion of a tubular shaft 700 illustrating various parameters of the tubular shaft 700. For example, a cut pattern of a tubular 700 may include parameters such as spine length 701; pitch 703; and columns 705 per revolution. Spine length 701 may define a length (parallel to a longitudinal axis 707) of a solid region between adjacent cuts ends 711a, 711b. Pitch 703 may define a length (parallel to a longitudinal axis 707 of the tubular shaft 700) between adjacent cuts 709a, 709b and can determine a stiffness of the tube. The tighter the pitch 703, the less distance between the adjacent cuts 709a, 709b, the tubular shaft may become more flexible. Column 705 per revolution can define the number of columns 705 per a 360 degree turn of the tubular shaft 700. Column 705 may reduce compression or elongation of the tubular shaft 700 when pushing the stent.

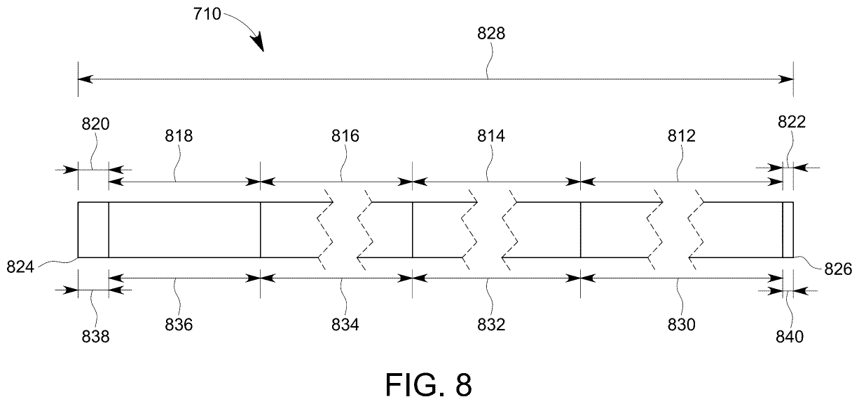

FIG. 8 shows a schematic of a proximal section 710 of the tubular shaft 700. The proximal section 710 may include one or more regions 812, 814, 816, 818, 820, and/or 822. In some embodiments, the proximal section 710 includes a plurality of regions (e.g., regions 812, 814, 816, 818, 820, 822). The proximal section 710 may include at least one region, for example region 812, region 814, region 816, region 818, region 820, or region 822.

In some embodiments, region 812 may have a length 830 that is about 80% to about 95% of a total length 828. Region 812 may have a length 830 that is about 85% to about 99% of a total length 828. Region 812 may have a length 830 that is about 85% to about 90% of a total length 828.

In some embodiments, region 814 may have a length 832 that is about 1% to about 10% of a total length 828; about 2% to about 8% of a total length 828; about 4% to about 6% of a total length 828; or about 5% to about 6% of a total length 828.

In some embodiments, region 816 may have a length 834 that is about 1% to about 10% of a total length 828; about 2% to about 6% of a total length 828; about 3% to about 6% of a total length 828; or about 4% to about 5% of a total length 828.

In some embodiments, region 818 may have a length 836 that is about 0.1% to about 2% of a total length 828; about 0.1% to about 1% of a total length 828; about 0.1% to about 0.5% of a total length 828; about 0.1% to about 1.5% of a total length 828; about 0.2% to about 0.3% of a total length 828; or about 0.2% to about 1% of a total length 828.

In some embodiments, regions 820, 822 may be an unmodified hypotube. Said another way, a distal end 824 and/or a proximal end 826 of proximal section 710 may be an unmodified hypotube. For example, region 820 may be used for bonding to an implant coupling section 720 or a distal section 730. In some embodiments, region 820 may have a length 838 that is about 0.01% to about 1% of a total length 828; about 0.01% to about 0.08% of a total length 828; about 0.01% to about 0.08% of a total length 828; about 0.02% to about 0.06% of a total length 828; about 0.03% to about 0.05% of a total length 828; about 0.005% to about 0.05% of a total length 828; or about 0.008% to about 0.015% of a total length 828.

In some embodiments, a total length 828 of a proximal section 710 may be about 75 cm to about 100 cm; about 75 cm to about 90 cm; or about 80 cm to about 90 cm, for example for pediatric applications. A total length 828 of a proximal section 710 may be about 120 cm to about 150 cm, for example for intracranial applications. A total length 828 of a proximal section 710 may be about 80 cm to about 140 cm, for example for vascular applications.

Any of regions 812, 814, 816, 818, 820, or 822 may have a cut pattern with a progressively increasing pitch. Any of regions 812, 814, 816, 818, 820, or 822 may have a cut pattern with a progressively decreasing pitch. Any of regions 812, 814, 816, 818, 820, or 822 may have a cut pattern with a substantially constant pitch.

In some embodiments, a region having a cut pattern with a progressively decreasing pitch is adjacent to a region having a cut pattern with a substantially constant pitch. In some embodiments, a region having a cut pattern with a progressively increasing pitch is adjacent to a region having a cut pattern with a substantially constant pitch. In some embodiments, a region having a cut pattern with a substantially constant pitch is in between a region having a cut pattern with a progressively changing pitch (either increasing or decreasing pitch).

Any of regions 812, 814, 816, 818, 820, or 822 may have a cut pattern with a progressively increasing spine length. Any of regions 812, 814, 816, 818, 820, or 822 may have a cut pattern with a progressively decreasing spine length. Any of regions 812, 814, 816, 818, 820, or 822 may have a cut pattern with a substantially constant spine length.

In some embodiments, a region having a cut pattern with a progressively decreasing spine length is adjacent to a region having a cut pattern with a substantially constant spine length. In some embodiments, a region having a cut pattern with a progressively increasing spine length is adjacent to a region having a cut pattern with a substantially constant spine length. In some embodiments, a region having a cut pattern with a substantially constant spine length is in between region having a cut pattern with a progressively changing spine length (either increasing or decreasing spine length).

Any of regions 812, 814, 816, 818, 820, or 822 may be substantially unmodified. Any of regions 812, 814, 816, 818, 820, or 822 may have an interrupted spiral cut pattern. Any of regions 812, 814, 816, 818, 820, or 822 may have a brick cut pattern.

In some embodiments, a region having a cut pattern with a progressively decreasing pitch is adjacent to a region that is substantially unmodified. In some embodiments, a substantially unmodified region is adjacent to a region having a cut pattern with a substantially constant pitch. In some embodiments, a substantially unmodified region is adjacent to region having an interrupted spiral cut pattern. In some embodiments, a substantially unmodified region is adjacent to a region having brick cut pattern. In some embodiments, an unmodified region is on a distal end 824 of the proximal section 710. In some embodiments, an unmodified region is on a proximal end 826 of the proximal section 710.

In some embodiments, a region having an interrupted spiral cut pattern is nonoverlapping with a region having a brick cut pattern. In some embodiments, a region having an interrupted spiral cut pattern is overlapping with a region having a brick cut pattern.

In some embodiments, at least one region has a cut pattern that has a progressively increasing pitch, increasing in a proximal direction toward a proximal end 826 of the proximal section 710. In some embodiments, one or more regions have a cut pattern that has a progressively increasing pitch, increasing in a proximal direction toward a proximal end 826 of the proximal section 710. The pitch may progressively increase from about 0.025 mm (0.001 in) to about 0.203 mm (0.008 in); about 0.051 mm (0.002 in) to about 0.203 mm (0.008 in); about 0.051 mm (0.002 in) to about 0.179 mm (0.007 in); about 0.051 mm (0.002 in) to about 0.152 mm (0.006 in); or about 0.076 mm (0.003 in) to about 0.152 mm (0.006 in). For example, any of regions 812, 814, 816, or 818 may have a progressively increasing pitch, increasing in a proximal direction toward a proximal end 826 of the proximal section 710. For example, region 814 may have a progressively increasing pitch, increasing in a proximal direction toward a proximal end 826 of the proximal section 710.

In some embodiments, at least one region has a cut pattern that has a progressively decreasing pitch, decreasing in a proximal direction toward a proximal end 826 of the proximal section 710. In some embodiments, one or more regions have a cut pattern that has a progressively decreasing pitch, decreasing in a proximal direction toward a proximal end 826 of the proximal section 710. The pitch may progressively decrease from about 0.203 mm (0.008 in) to about 0.025 mm (0.001 in); about 0.203 mm (0.008 in) to about 0.051 mm (0.002 in); about 0.179 mm (0.007 in) to about 0.051 mm (0.002 in); about 0.152 mm (0.006 in) to about 0.051 mm (0.002 in); or about 0.152 mm (0.006 in) to about 0.076 mm (0.003 in). For example, any of regions 812, 814, 816, or 818 may have a progressively decreasing pitch, decreasing in a proximal direction toward a proximal end 826 of the proximal section 710. For example, region 818 may have a progressively decreasing pitch, decreasing in a proximal direction toward a proximal end 826 of the proximal section 710.

In some embodiments, at least one region has a cut pattern that has a substantially constant pitch. In some embodiments, one or more regions have a cut pattern that has a substantially constant pitch. In some embodiments, the substantially constant pitch is between about 0.025 mm (0.001 in) to about 0.38 mm (0.015 in); about 0.025 mm (0.001 in) to about 0.152 mm (0.006 in); about 0.051 mm (0.002 in) to about 0.127 mm (0.005 in); about 0.051 mm (0.002 in) to about 0.102 mm (0.004 in); about 0.051 mm (0.002 in) to about 0.076 mm (0.003 in); about 0.203 mm (0.008 in) to about 0.38 mm (0.015 in); about 0.203 mm (0.008 in) to about 0.305 mm (0.012 in); about 0.229 mm (0.009 in) to about 0.280 mm (0.011 in); or 0.229 mm (0.009 in) to about 0.254 mm (0.010 in). For example, any of regions 812, 814, 816, or 818 may have a substantially constant pitch. For example, one or both of region 812 or region 816 may have a substantially constant pitch. For example, one or both of region 812 or region 816 may have a substantially constant pitch between about 0.203 mm (0.008 in) to about 0.305 mm (0.012 in). For example, one or both of region 812 or region 816 may have a substantially constant pitch between about 0.229 mm (0.009 in) to about 0.280 mm (0.011 in).

In some embodiments, at least one region of the proximal section 710 has a plurality of relief cuts that are formed in an interrupted spiral cut pattern. For example, about 5% to about 99% of the proximal section 710 has an interrupted spiral cut pattern. About 10% to about 95% of the proximal section 710 may have an interrupted spiral cut pattern. About 50% to about 95% of the proximal section 710 may have an interrupted spiral cut pattern. About 60% to about 95% of the proximal section 710 may have an interrupted spiral cut pattern. About 80% to about 95% of the proximal section 710 may have an interrupted spiral cut pattern. About 60% to about 99% of the proximal section 710 may have an interrupted spiral cut pattern. About 70% to about 90% of the proximal section may have an interrupted spiral cut pattern.

For example, one of the proximal most regions has an interrupted spiral cut pattern. A region that is within about 60 cm to about 80 cm of the proximal end 826 may have an interrupted spiral cut pattern. A region that is within about 70 cm to about 80 cm of the proximal end 826 may have an interrupted spiral cut pattern. A region that is within about 75 cm to about 85 cm of the proximal end 826 may have an interrupted spiral cut pattern. A region that is within about 75 cm to about 80 cm of the proximal end 826 may have an interrupted spiral cut pattern. For example, any of regions 812, 814, 816, or 818 may have an interrupted spiral cut pattern. For example, region 812 may have an interrupted spiral cut pattern.

In some embodiments, the interrupted spiral cut pattern may have about 1.5 columns per revolution (CPR) to about 4.5 CPR. The interrupted spiral cut pattern may have about 2 CPR to about 4 CPR. The interrupted spiral cut pattern may have about 2 CPR to about 3 CPR. The interrupted spiral cut pattern may have about 2.5 CPR. The CPR may be substantially constant. The CPR may progressively increase from a distal end 824 of the proximal section 710 to a proximal end 826 of the proximal section 710. The CPR may progressively decrease from a distal end 824 of the proximal section 710 to a proximal end 826 of the proximal section 710.

In some embodiments, the interrupted spiral cut pattern may have about 40 degrees to about 80 degrees uncut (i.e., about 40 degrees to about 80 degrees of a 360-degree revolution remains uncut). The interrupted spiral cut pattern may have about 50 degrees to about 70 degrees uncut. The interrupted spiral cut pattern may have about 55 degrees to about 65 degrees uncut.

In some embodiments, at least one region of the proximal section 710 has a plurality of relief cuts that are formed in brick cut pattern. For example, about 5% to about 99% of the proximal section 710 has a brick cut pattern. About 10% to about 95% of the proximal section 710 may have a brick cut pattern. About 5% to about 30% of the proximal section 710 may have a brick cut pattern. About 5% to about 20% of the proximal section 710 may have a brick cut pattern. About 3% to about 20% of the proximal section 710 may have a brick cut pattern. About 7% to about 15% of the proximal section 710 may have a brick cut pattern. About 8% to about 12% of the proximal section 710 may have a brick cut pattern. In some embodiments, one or more of the distal most regions of the proximal section 710 has a brick cut pattern. In some embodiments, a region, or one or more regions, that is within about 0.05 cm to about 8 cm of the distal end 824 has a brick cut pattern. A region that is within about 2 cm to about 8 cm of the distal end 824 may have a brick cut pattern. A region that is within about 2 cm to about 4 cm of the distal end 824 may have a brick cut pattern. A region that is within about 4 cm to about 6 cm of the distal end 824 may have a brick cut pattern. A region that is within about 0.05 cm to about 2 cm of the distal end 824 may have a brick cut pattern. For example, any of regions 812, 814, 816, or 818 may have a brick cut pattern. For example, one or more of regions 814, 816, or 818 may have a brick cut pattern.

In some embodiments, at least one region has a cut pattern that has a progressively increasing spine length, increasing in a proximal direction toward a proximal end 826 of the proximal section 710. For example, the spine length may increase from about 0.051 mm (0.002 in) to about 0.254 mm (0.010 in). The spine length may increase from about 0.076 mm (0.003 in) to about 0.254 mm (0.010 in). The spine length may increase from about 0.101 mm (0.004 in) to about 0.254 mm (0.010 in). The spine length may increase from about 0.051 mm (0.002 in) to about 0.229 mm (0.009 in). The spine length may increase from about 0.127 mm (0.005 in) to about 0.203 mm (0.008 in). For example, any of regions 812, 814, 816, or 818 may have a progressively increasing spine length, increasing in a proximal direction toward a proximal end 826 of the proximal section 710. For example, region 814 may have a progressively increasing spine length, increasing in a proximal direction toward a proximal end 826 of the proximal section 710.

In some embodiments, at least one region has a cut pattern that has a progressively decreasing spine length, decreasing in a proximal direction toward a proximal end 826 of the proximal section 710. For example, the spine length may decrease from about 0.254 mm (0.010 in) to about 0.051 mm (0.002 in). The spine length may decrease from about 0.254 mm (0.010 in) to about 0.076 mm (0.003 in). The spine length may decrease from about 0.254 mm (0.010 in) to about 0.101 mm (0.004 in). The spine length may decrease from about 0.229 mm (0.009 in) to about 0.051 mm (0.002 in). The spine length may decrease from about 0.203 mm (0.008 in) to about 0.127 mm (0.005 in). For example, any of regions 812, 814, 816, or 818 may have a progressively decreasing spine length, decreasing in a proximal direction toward a proximal end 826 of the proximal section 710. For example, region 818 may have a progressively decreasing spine length, decreasing in a proximal direction toward a proximal end 826 of the proximal section 710.

In some embodiments, at least one region has a cut pattern that has a substantially constant spine length. The substantially constant spine length may be between about 0.051 mm (0.002 in) to about 0.203 mm (0.008 in). The substantially constant spine length may be between about 0.051 mm (0.002 in) to about 0.178 mm (0.007 in). The substantially constant spine length may be between about 0.076 mm (0.003 in) to about 0.178 mm (0.007 in). The substantially constant spine length may be between about 0.102 mm (0.004 in) to about 0.152 mm (0.006 in). For example, any of regions 812, 814, 816, or 818 may have a substantially constant spine length. For example, region 816 may have a substantially constant spine length.

In some embodiments, a region, for example any of regions 812, 814, 816, 818, may have a progressively decreasing pitch and a progressively decreasing spine length. For example, region 818 may have a progressively decreasing pitch and a progressively decreasing spine length. A combination of a progressively decreasing pitch and a progressively decreasing spine length, decreasing in a proximal direction toward a proximal end 826 of the proximal section 710, may result in decreased flexibility toward the distal end 824 of the proximal section 710. Decreasing flexibility toward the distal end 824 of the proximal section 710 may be desirable to transition from the proximal section to the implant coupling section 720 and/or the distal section 730. In contrast, substantially constant pitch or increasing pitch, toward a proximal end, in at least some of the regions of the proximal section, for example regions 816, 814 may increase flexibility and therefore be desirable for tracking the tubular shaft to a site in the vasculature. A tubular shaft may be navigated through tortuous anatomy, for example vasculature at or surrounding the ductus arteriosus or intracranially. Further, as will be described in further detail elsewhere herein, flexibility in a distal end of the tubular shaft, in particular the implant receiving section 732, may be desirable for tracking in such tortuous anatomy.

In some embodiments, a region, for example any of regions 812, 814, 816, 818, may have a substantially constant pitch. A substantially constant pitch may increase column strength and/or pushability of the tubular shaft. Pushability and column strength may be desirable in a proximal portion of the proximal section 710, for example region 812, to be able to push the system through the catheter and through the vasculature to a target site.

In some embodiments, a region, for example any of regions 812, 814, 816, 818, may have a substantially constant pitch and an interrupted spiral pattern. A combination of a substantially constant pitch and an interrupted spiral pattern may increase column strength and/or pushability of the tubular shaft. Pushability and column strength may be desirable in a proximal portion of the proximal section 710, for example region 812, to be able to push the system through the catheter and through the vasculature to a target site.

In some embodiments, a region, for example any of regions 812, 814, 816, 818, may have a substantially constant pitch and a CPR of about 1.5 to about 4.5. A combination of a substantially constant pitch and a particular CPR may increase column strength and/or pushability of the tubular shaft. Pushability and column strength may be desirable in a proximal portion of the proximal section 710, for example region 812, to be able to push the system through the catheter and through the vasculature to a target site.

In some embodiments, a region, for example any of regions 812, 814, 816, 818, may have a substantially constant pitch and a percent uncut of about 40% to about 60%. A combination of a substantially constant pitch and a particular percent uncut may increase column strength and/or pushability of the tubular shaft. Pushability and column strength may be desirable in a proximal portion of the proximal section 710, for example region 812, to be able to push the system through the catheter and through the vasculature to a target site.

In some embodiments, a region, for example any of regions 812, 814, 816, 818, may have a CPR of about 1.5 to about 4.5 and a percent uncut of about 40% to about 60%. A combination of a particular CPR and a particular percent uncut may increase column strength and/or pushability of the tubular shaft. Pushability and column strength may be desirable in a proximal portion of the proximal section 710, for example region 812, to be able to push the system through the catheter and through the vasculature to a target site.

In some embodiments, a region, for example any of regions 812, 814, 816, 818, may have an interrupted spiral cut pattern and a percent uncut of about 40% to about 60%. A combination of an interrupted spiral cut pattern and a particular percent uncut may increase column strength and/or pushability of the tubular shaft. Pushability and column strength may be desirable in a proximal portion of the proximal section 710, for example region 812, to be able to push the system through the catheter and through the vasculature to a target site.

In some embodiments, a region, for example any of regions 812, 814, 816, 818, may have an interrupted spiral cut pattern and a CPR of about 1.5 to about 4.5. A combination of an interrupted spiral cut pattern and a particular CPR may increase column strength and/or pushability of the tubular shaft. Pushability and column strength may be desirable in a proximal portion of the proximal section 710, for example region 812, to be able to push the system through the catheter and through the vasculature to a target site.

In some embodiments, an outer diameter of the proximal section 710 is greater than an outer diameter of the distal section 730. For example, the implant is loaded on at least a portion of the distal section 730, so that distal section 730 is positioned in a lumen of the implant. In contrast, the proximal section 710 may be used to push the implant on the tubular shaft. As such, the proximal section 710 may have a substantially same outer diameter as an outer diameter of a crimped implant or unexpanded implant. In embodiments not having a distal section 730, an outer diameter of the proximal section 710 may be substantially similar to an outer diameter of the implant coupling section 720.

In some embodiments, an outer diameter of the proximal section 710 is sized to be received within a lumen of a catheter, for example a microcatheter. In some embodiments, an outer diameter of a proximal section is about 0.055 cm (0.022 in) to about 0.085 cm (0.033 in). In some embodiments, an outer diameter of a proximal section is about 0.060 cm (0.024 in) to about 0.070 cm (0.028 in). In some embodiments, an inner diameter of the proximal section 710 is sized to receive a guidewire therethrough. In some embodiments, an inner diameter of the proximal section 710 may be at least about 0.038 cm. An inner diameter of the proximal section 710 may be about 0.038 cm (0.015 in) to about 0.050 cm (0.020 in). An inner diameter of the proximal section 710 may be about 0.045 cm (0.018 in) to about 0.052 cm (0.020 in). An inner diameter of the proximal section 710 may be about 0.046 cm (0.018 in) to about 0.050 cm (0.020 in).

In some embodiments, a wall thickness of the proximal section is about 0.127 mm (0.005 in) to about 0.254 mm (0.01 in). In some embodiments, a wall thickness of the proximal section is about 0.1524 mm (0.006 in) to about 0.229 mm (0.009 in). In some embodiments, a wall thickness of the proximal section is about 0.178 mm (0.007 in) to about 0.229 mm (0.009 in).

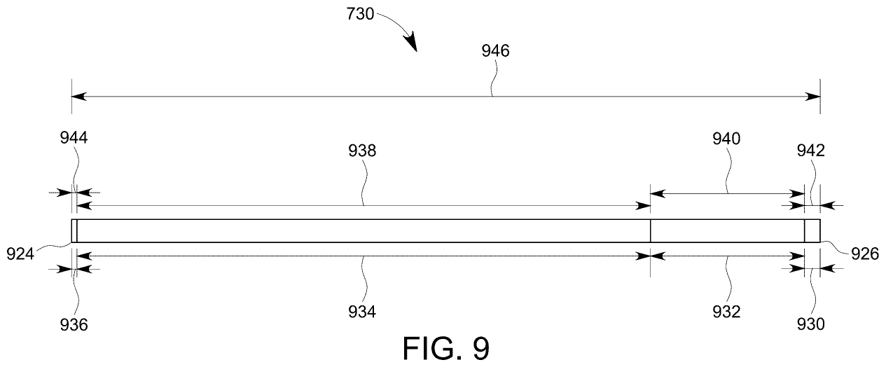

FIG. 9 shows a distal section 730 of a tubular shaft 700. The distal section 730 includes one or more regions 938, 940, 942, 944. In some embodiments, the distal section 730 includes a plurality of regions 938, 940, 942, 944. In some embodiments, the distal section 730 includes at least one region, for example region 938, region 940, region 942, or region 944.

In some embodiments, region 938 may have a length 934 that is about 60% to about 85% of a total length 946. Region 938 may have a length 934 that is about 65% to about 85% of a total length 946. Region 938 may have a length 934 that is about 70% to about 85% of a total length 946. Region 938 may have a length 934 that is about 75% to about 80% of a total length 946.

In some embodiments, region 940 may have a length 932 that is about 10% to about 30% of a total length 946. Region 940 may have a length 932 that is about 15% to about 30% of a total length 946. Region 940 may have a length 932 that is about 15% to about 25% of a total length 946. Region 940 may have a length 932 that is about 18% to about 22% of a total length 946.

In some embodiments, region 942 may have a length 930 that is about 0.5% to about 5% of a total length 946. Region 942 may have a length 930 that is about 1% to about 3% of a total length 946. Region 942 may have a length 930 that is about 1.5% to about 2.5% of a total length 946.

In some embodiments, region 944 may have a length 936 that is about 0.1% to about 1% of a total length 946. Region 944 may have a length 936 that is about 0.25% to about 1% of a total length 946. Region 944 may have a length 936 that is about 0.5% to about 0.8% of a total length 946. Region 944 may have a length 936 that is about 0.5% to about 0.75% of a total length 946.

A total length 946 of distal section 730 may be about 1 cm to about 3 cm. A total length 946 of distal section 730 may be about 1.5 cm to about 2.5 cm. A total length 946 of distal section 730 may be about 1.5 cm to about 2 cm. A total length 946 of distal section 730 may be about 1.8 cm to about 2 cm.

In some embodiments, one or both of regions 942, 944 may be an unmodified hypotube. For example, a proximal end 926 of region 942 may couple or be bonded to an implant coupling section 720, as shown in FIG. 7A.

In some embodiments, one or more regions, for example any of region 938, 940, 942, 944 include a plurality of relief cuts that are formed in an interrupted spiral cut pattern. The interrupted spiral cut pattern may be, at least partially, characterized by a progressively decreasing pitch, decreasing in a proximal direction toward a proximal end 926 of the distal section 730. The pitch may be decrease from about 0.152 mm (0.006 in) to about 0 0.025 mm (0.001 in). The pitch may decrease from about 0.127 mm (0.005 in) to about 0.051 mm (0.002 in). The pitch may decrease from about 0.102 mm (0.004 in) to about 0.076 mm (0.003 in). A progressively decreasing pitch, decreasing in a proximal direction, may be desirable to transition from a less flexible implant coupling section 720 to a more flexible distal section 730, with an implant receiving section 732, as shown in FIG. 7A. Said another way, one or more proximal regions (e.g., adjacent proximal end 926) of the distal section 730 and one or more distal regions (e.g., adjacent distal end 824) of the proximal section 710 may be less stiff than the implant coupling section 720 but more stiff than one or more distal regions of the distal section 730 and one or more proximal regions of the proximal section 710 to transition from the less flexible implant coupling section 720 to the more flexible regions of the proximal section 710 and distal section 730. In some embodiments, any of regions 938, 940, 942, or 944 may have a progressively decreasing pitch. In some embodiments, region 940 may have a progressively decreasing pitch.

The interrupted spiral cut pattern may be, at least partially, characterized by a progressively increasing pitch, increasing in a proximal direction toward a proximal end 926 of the distal section 730. The pitch may be increase from about 0.025 mm (0.001 in) to about 0.152 mm (0.006 in). The pitch may increase from about 0.051 mm (0.002 in) to about 0.127 mm (0.005 in). The pitch may increase from about 0.076 mm (0.003 in) to about 0.102 mm (0.004 in).

The interrupted spiral cut pattern may be, at least partially, characterized by a substantially constant pitch. The substantially constant pitch may be between about 0.025 mm (0.001 in) to about 0.127 mm (0.005 in). The substantially constant pitch may be between about 0.051 mm (0.002 in) to about 0.102 mm (0.004 in). The substantially constant pitch may be between about 0.076 mm. A substantially constant pitch may provide flexibility in the region while also providing column strength for tracking or maintaining support for an implant positioned about the region. In some embodiments, any of regions 938, 940, 942, or 944 may have a substantially constant pitch. In some embodiments, region 938 may have a substantially constant pitch. The substantially constant pitch near a distal end 924 (e.g., region 944) of the distal section 730 may provide flexibility for tracking in tortuous vasculature while maintaining support for an implant positioned at least partially on or about the region, for example, region 938.

In some embodiments, one or more regions, for example any of regions 938, 940, 942, or 944, may include a substantially constant, progressively increasing (toward a proximal end 926), or progressively decreasing (toward a proximal end 926) CPR. For example, the CPR may be between about 0.5 to about 4.5. The CPR may be between about 1 to about 4. The CPR may be between about 1.5 to about 2.5. The CPR may be substantially about 1.5. For example, one or both of regions 938 and 940 may include a substantially constant CPR.

In some embodiments, one or more regions, for example any of regions 938, 940, 942, or 944, may include a substantially constant, progressively increasing (toward a proximal end 926), or progressively decreasing (toward a proximal end 926) number of degrees that remain uncut. For example, the number of degrees that remain uncut may be between about 10 degrees to about 40 degrees. The number of degrees that remain uncut may be between about 10 degrees to about 30 degrees. The number of degrees that remain uncut may be between about 15 degrees to about 30 degrees. The number of degrees that remain uncut may be between about 15 degrees to about 25 degrees. The number of degrees that remain uncut may be between about 10 degrees to about 20 degrees. The number of degrees that remain uncut may be between about 15 degrees to about 17 degrees. The number of degrees that remain uncut may be substantially about 16 degrees. For example, one or both of regions 938 and 940 may include a substantially constant number of degrees that remain uncut.

In some embodiments, a number of degrees that remain uncut may progressively decrease (decrease towards a proximal end 926 of the distal section 730). For example, the number of degrees that remain uncut may progressively decrease from about 40 degrees to about 10 degrees. The number of degrees that remain uncut may progressively decrease from about 30 degrees to about 10 degrees. The number of degrees that remain uncut may progressively decrease from about 25 degrees to about 10 degrees. The number of degrees that remain uncut may progressively decrease from about 25 degrees to about 20 degrees. The number of degrees that remain uncut may progressively decrease from about 30 degrees to about 15 degrees. The number of degrees that remain uncut may progressively decrease from about 25 degrees to about 16 degrees. Any one of regions 938, 940, 942, or 944 may include a pattern that includes a progressively decreasing number of degrees that remain uncut. For example, region 940 may have a pattern that includes a progressively decreasing number of degrees that remain uncut.

In some embodiments, a number of degrees that remain uncut may progressively increase (increase towards a proximal end 926 of the distal section 730). For example, the number of degrees that remain uncut may progressively increase from about 10 degrees to about 40 degrees. The number of degrees that remain uncut may progressively increase from about 10 degrees to about 30 degrees. The number of degrees that remain uncut may progressively increase from about 10 degrees to about 25 degrees. The number of degrees that remain uncut may progressively increase from about 20 degrees to about 25 degrees. The number of degrees that remain uncut may progressively increase from about 15 degrees to about 30 degrees. The number of degrees that remain uncut may progressively increase from about 16 degrees to about 25 degrees. Any one of regions 938, 940, 942, or 944 may include a pattern that includes a progressively increasing number of degrees that remain uncut. For example, region 940 may have a pattern that includes a progressively increasing number of degrees that remain uncut.

FIGS. 10A-12 show various embodiments of implant coupling sections. Each implant coupling section 720 defines a lumen for receiving a guidewire therethrough. The lumen of the implant coupling section 720 may have an inner diameter that is similar to or greater than a guidewire outer diameter. For example, the lumen of the implant coupling section 720 may have an inner diameter than is greater than about 0.0381 cm (0.015 in). For example, an inner diameter of the lumen of the implant coupling section 720 may be between about 0.0406 cm (0.016 in) to about 0.0508 cm (0.02 in).

FIG. 10A shows an embodiment of an implant coupling section 720a. Implant coupling section 720a defines a lumen 1012 and optionally includes one or more bumpers or backstops 1010 for pushing an implant at least partially carried by a tubular shaft. For example, one or more radiopaque markers or features of an implant may be coupled to or press fit into a window or aperture 1016 defined by the implant coupling section 720a. The bumper or backstop 1010 may protrude from a surface 1018, for example in a vertical plane, of the implant coupling section 720a. As an implant is navigated distally on a tubular shaft through the catheter, forces may push the implant proximally along the tubular shaft. The bumper or backstop 1010 interacts with the one or more radiopaque markers or features to reduce or prevent travel of the implant proximally along the implant coupling section 720a, and therefore the tubular shaft. This may occur with a multi-section tubular shaft, as shown in FIG. 7A. Additionally, or alternatively, this may occur with a tubular shaft that does not include a distal section 730. Additionally, or alternatively, this may occur with a tubular shaft having a monolithic construction where an outer diameter of the tubular shaft does not substantially change and/or where an outer diameter of the tubular shaft substantially matches an inner diameter of the implant thereon or an inner diameter of the catheter in which the implant is traveling through. Each window or aperture 1016 may include a respective backstop 1010 on a proximal end 1020 (as opposed to a distal end 1022) of the window or aperture 1016. Alternatively, one or more or a subset of the apertures 1016 may include a backstop 1010, while a subset does not include a backstop 1010.

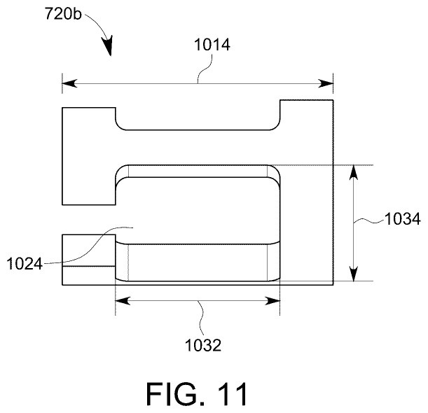

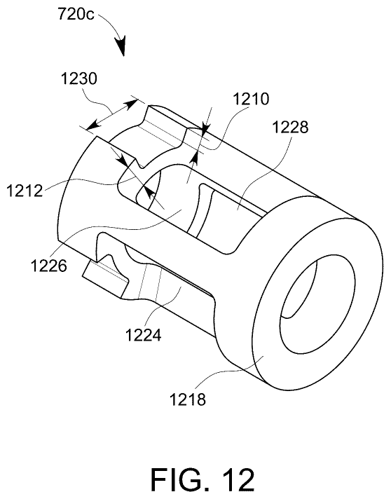

FIGS. 10B-12 show an implant coupling section 720b having a similar structure as shown above in FIG. 10A, except that the implant coupling section 720b of FIGS. 10B-12 does not include the backstop 1010. FIGS. 10B-12 show an implant receiving section 720b defining a plurality of apertures, aperture 1024, aperture 1026, and aperture 1028. Each aperture 1024, 1026, 2028 includes a respective channel 1030 for receiving a tab of an implant. For example, FIGS. 13-14 show a tab 1316 of an implant terminating at a radiopaque marker 1300. The radiopaque marker 1300 is press fit into an aperture 1314 defined by an implant coupling section 720d. The tab 1316 is press fit into the channel 1312 or indentation (described below in connection with FIG. 12). The implant coupling section 720d is bonded to the proximal section 1310. The implant 1318 overlays at least a portion of the distal section that is also bonded to the implant coupling section 720d on an end opposite the proximal section 710. The channel 1030 extends from a distal end 1036 of the implant coupling section 720b to the aperture 1026. As shown in FIG. 11, the length 1032 (longitudinal length from proximal to distal) of the aperture, for example aperture 1024, is about 40% to about 80% of a length 1014 of the implant coupling section 720b. The length 1032 of the aperture 1024 may be about 50% to about 70% of a length 1014 of the implant coupling section 720b. In some embodiments, an aperture of the tubular shaft may be about twice as long as a length of the radiopaque marker to enable loading the tubular shaft into an inner diameter of the implant after crimping the implant. In some configurations, the implant coupling section 720 includes one or more apertures and the distal section 730 includes one or more apertures. In such embodiments, one or more apertures in the distal section 730 of the tubular shaft 700 may be longer than one or more proximal apertures to allow for length tolerance of an implant. A width 1034 of an aperture, for example, aperture 1024, may extend circumferentially along the implant receiving section 720b for about 45 degrees to about 110 degrees, for example for three apertures in an implant coupling section 720.

FIG. 12 shows an implanting receiving section 720c having a similar structure as shown above in FIGS. 10B-11, except FIG. 12 shows an indentation 1230 instead of a channel 1030. Indentation 1230 is shaped to receive a tab of an implant, and aperture (any of apertures 1224, 1226, 1228) is shaped to receiving a radiopaque marker or other feature of an implant. For example, a radiopaque marker or other feature may be press fit into aperture 1224, aperture 1226, and/or aperture 1228. A depth 1210 of the indentation 1230 may be about 40% to about 70% of a thickness 1212 of the sidewall 1218 of the implant coupling section 720c.

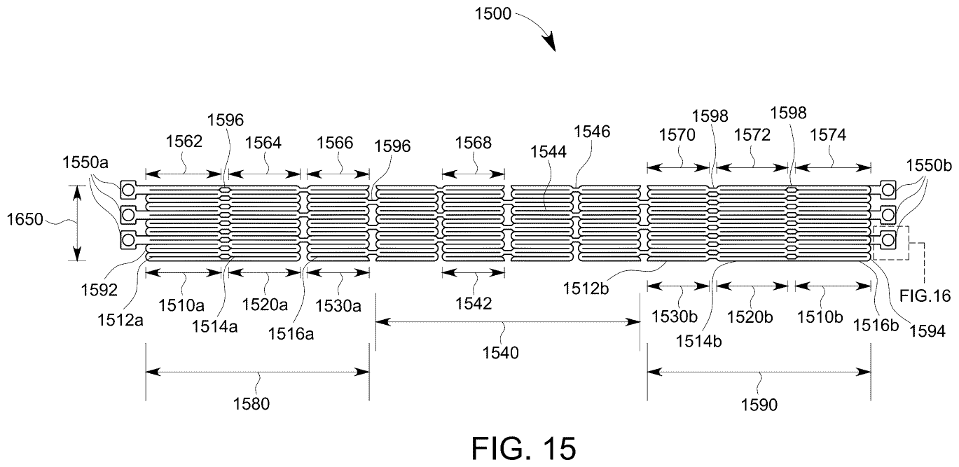

FIG. 15 shows an embodiment of an implant, for example a stent (e.g., implant 1500), in a 2D crimped configuration. As shown, the implant 1500 has a first end section 1580 having a distal end 1592, a second end section 1590 having a proximal end 1594, and a body section 1540 between the first end section 1580 and the second end section 1590.

The first end section 1580 includes one ring, more than one ring, or a plurality of rings. As shown in this embodiment, first end section 1580 includes a terminal ring 1510a including a plurality of struts 1512a, each having a length 1562; a penultimate ring 1520a including a plurality of struts 1514a, each having a length 1564; and an antepenultimate ring 1530a including a plurality of struts 1516a, each having a length 1566. Length 1562 of each strut 1512a may be substantially similar to length 1564 of each strut 1514a and/or length 1566 of each strut 1516a. Preferably, length 1562 is greater than length 1564 which is greater than length 1566, such that the lengths of the struts increase moving from the body section 1540 to the first end section 1580. In other embodiments, length 1566 is greater than length 1564 which is greater than length 1562, such that the lengths of the struts decrease moving from the body section 1540 to the first end section 1580. In a further iteration, length 1564 and 1566 may be substantially the same or length 1562 and 1564 may be substantially the same or length 1562 and 1566 may be substantially the same. Strut lengths 1562, 1564, and 1566 may each be between about 1.0 mm and about 2.5 mm. A length 1562 of each strut 1512a is about 1.5 mm to about 2.5 mm; or about 1.8 mm to about 2.2 mm. A length 1564 of each strut 1514a is about 1.5 mm to about 2.0 mm; or about 1.6 mm to about 1.9 mm. A length 1566 of each strut 1516a is about 1.0 mm to about 2.0 mm; or about 1.3 mm to about 1.7 mm. As shown in FIG. 15, a distal end 1592 of the first end section 1580 includes one, one or more, or a plurality of radiopaque markers 1550a. Alternatively, radiopaque markers 1550a may be replaced with a connecting element, such as a male or female connector that is configured to connect with a complementary feature (e.g., a female or male connector, respectively) on a delivery system or an implant coupling section 720. Rings 1510a and 1520a and rings 1520a and 1530a are connected to each other via one or more or a plurality of bridges 1596. As shown in FIG. 15, there may be about three bridges to about nine bridges.

The second end section 1590 included one ring, more than one ring, or a plurality of rings. As shown in this embodiment, second end section 1590 includes a terminal ring 1510b including a plurality of struts 1516b, each having length 1574; a penultimate ring 1520b including a plurality of struts 1514b, each having a length 1572; and an antepenultimate ring 1530b including a plurality of struts 1512b, each having a length 1570. Length 1574 of each strut 1516b may be substantially similar to length 1572 of each strut 1514b and/or length 1570 of each strut 1512b. Preferably, length 1574 is greater than length 1572, which is greater than length 1570 of each strut 1512b, such that the lengths of the struts increase moving from the body section 1540 to the second end section 1590. In other embodiments, length 1570 is greater than length 1572, which is greater than length 1574, such that the lengths of the struts decrease moving from the body section 1540 to the second end section 1590. In a further variation, length 1574 and 1572 may be substantially the same or length 1574 and 1570 may be substantially the same or length 1572 and 1570 may be substantially the same. Strut lengths 1572, 1574, and 1570 may each be between about 1.0 mm and about 2.5 mm. A length 1574 of each strut 1516b is about 1.5 mm to about 2.5 mm; or about 1.8 mm to about 2.2 mm. A length 1572 of each strut 1514b is about 1.5 mm to about 2.0 mm; or about 1.6 mm to about 1.9 mm. A length 1570 of each strut 1512b is about 1.0 mm to about 2.0 mm; or about 1.3 mm to about 1.7 mm. As shown in FIG. 15, a proximal end 1594 includes one, one or more, or a plurality of radiopaque markers 1550b. Alternatively, radiopaque markers 1550b may be replaced with a connecting element, such as a male or female connector that is configured to connect with a complementary feature (e.g., a female or male connector, respectively) on a delivery system, tubular shaft, or an implant coupling section 720. Rings 1510b and 1520b and rings 1520b and 1530b are connected to each other via one or more or a plurality of bridges 1598. As shown in FIG. 15, there may be about three bridges to about nine bridges between each pair of adjacent rings.

Body section 1540 includes a plurality of rings 1542, each including a plurality of struts 1544. Body section 1540 may include one ring or one or more rings (e.g., in a septal defect embodiment) or more than one ring or a plurality of rings (e.g., in a patent ductus arteriosus embodiment). For example, there may be about one ring, about 2 to about 6 rings, or about 3 to about 10 rings. The plurality of struts 1544 of the body section 1540 each have a length 1568. As shown in FIG. 15, the length 1568 of each of the struts 1544 may be about 1.0 mm to about 2.0 mm, preferably about 1.4 mm to about 1.7 mm. The rings 1542 of the body section 1540 may be connected via a plurality of bridges 1546, for example about three bridges to about nine bridges between each pair of adjacent rings. The implant may be a Nitinol stent, a braided Cobalt Chromium stent, a polymer stent, or another braided design. The implant could be used in a variety of cardiovascular non-balloon-expandable stent delivery applications including peripheral vascular, coronary, and neurovascular applications.

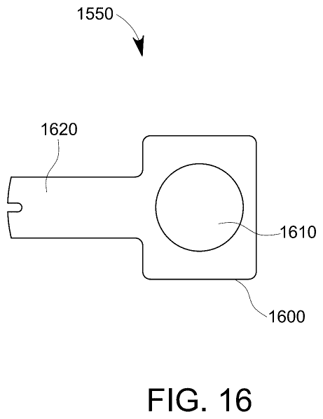

FIG. 16 shows a zoomed in view of an embodiment of a radiopaque marker of an implant. The radiopaque marker 1550 includes a metal perimeter 1600 that contains a radiopaque region 1610. The radiopaque region 1610 may be sized so that it is above a threshold detection level of a measuring instrument (fluoroscope). For example, the radiopaque region 1610 may have a diameter of about 5 mm to about 10 mm. The metal perimeter 1600 is coupled to, or bonded to, a tab 1620 that extends radially from a distal end 1592 and/or a proximal end 1594 of the implant 1500. The tab 1620 may be press fit into the channel 1030 of FIG. 10B or the indentation 1230 of FIG. 12. The metal perimeter 1600 may be press fit into the any of apertures 1024, 1026, 1028 (FIG. 10B) or apertures 1224, 1226, 1226 (FIG. 12). In some examples, an implant may include three radiopaque markers per end (e.g., three markers on a proximal end and three markers on distal end). In some embodiments, about two radiopaque markers per end of an implant can be engaged with the tubular shaft to enable for substantially similar advancing and retracting of the implant within the catheter and/or relative to the catheter.

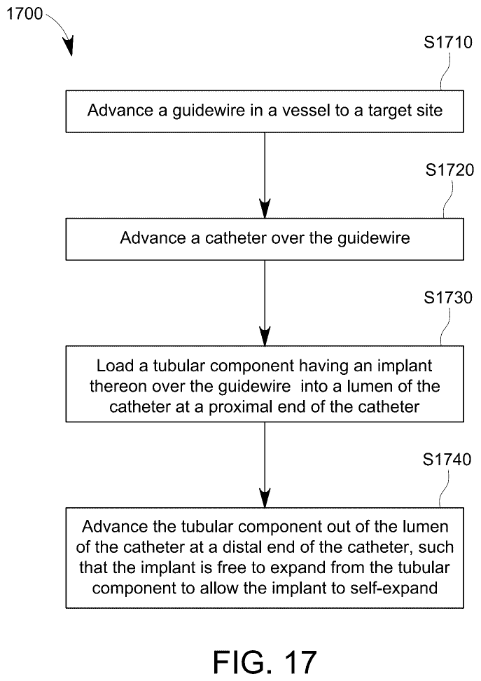

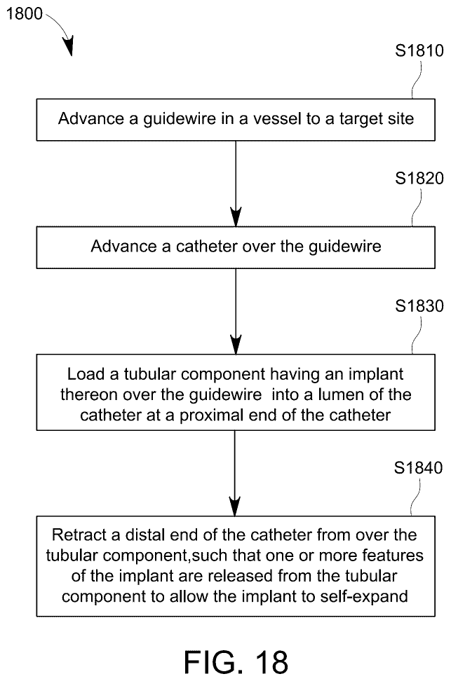

Turning now to FIGS. 17-18. In general, the methods described herein enable an operator to gain access to a treatment vessel with a guidewire and catheter selected by the operator. The guidewire can be “back-loaded” into the tubular shaft in a pre-loaded tubular shaft-implant-transfer sheath complex. The tubular shaft may be used to push the implant over the loaded guidewire out of the transfer sheath and into the catheter. This allows the operator to use the guidewire and an operator-selected catheter rather than a full implant delivery system. The implant is then delivered by pushing or advancing the tubular shaft-implant complex up to the desired treatment location (through a catheter lumen) and then cither pushing (urging or advancing) the implant out of the catheter or pulling (or retracting) the catheter back to unsheathe the implant. Once the implant is out of the catheter, the radiopaque markers disengage from the windows of the tubular shaft upon self-expansion.

FIG. 17 is a flow chart of an embodiment of a method 1700 of delivering an implant to a target site. The method 1700 includes: advancing a guidewire in a vessel to a target site at block S1710; advancing a catheter over the guidewire at block S1720; loading a tubular shaft having an implant thereon over the guidewire into a lumen of the catheter at a proximal end of the catheter at block S1730; and advancing the tubular shaft out of the lumen of the catheter at a distal end of the catheter, such that one or more features of the implant are released from the tubular shaft to allow the implant to self-expand at block S1740. The method 1700 functions to use access of an existing guidewire and catheter to position an implant, such that the implant delivery system can be backloaded into the catheter (at a proximal end or user end of the catheter) and over the guidewire. Alternatively, a guidewire may be parked, and a differently sized or shaped catheter (e.g., based on physician's choice) may be advanced over the guidewire and then the tubular shaft and implant backloaded into the catheter and delivered to the target site. In the method 1700 of FIG. 17, the distal end of the catheter and/or guidewire) may be substantially maintained (parked) at a position while the tubular shaft and implant are advanced out of the distal end to release the implant.

FIG. 18 is a flow chart of an embodiment of a method 1800 of delivering an implant to a target site. The method 1800 includes: advancing a guidewire in a vessel to a target site at block S1810; advancing a catheter over the guidewire at block S1820; loading a tubular shaft having an implant thereon over the guidewire into a lumen of the catheter at a proximal end of the catheter at block S1830; and retracting a distal end of the catheter from over the tubular shaft, such that one or more features of the implant are released from the tubular shaft to allow the implant to self-expand at block S1840. The method 1800 functions to use access of an existing guidewire and catheter to position an implant, such that the implant delivery system can be backloaded into the catheter (at a proximal end or user end of the catheter) and over the guidewire. Alternatively, a guidewire may be parked, and a differently sized or shaped catheter (e.g., based on physician's choice) may be advanced over the guidewire and then the tubular shaft and implant backloaded into the catheter and delivered to the target site. In the method 1800 of FIG. 18, the distal end of the tubular shaft (with the implant thereon) may be substantially maintained at a position while the distal end of the catheter is retracted to release the implant from the tubular shaft.

### Examples

Example 1. A system for delivering a vascular implant, the system comprising: a tubular shaft defining a lumen and comprising: a proximal section, an implant coupling section bonded to the proximal section, wherein the implant coupling section defines at least one aperture, and wherein the at least one aperture is configured to receive a feature of an implant positioned on an outer diameter of the tubular shaft, and a plurality of relief cuts in the tubular shaft to increase a flexibility of the tubular shaft.

Example 2. The system of any one of the preceding Examples, but particularly Example 1, further comprising a distal section, wherein the implant coupling section is between the proximal section and the distal section, and wherein the distal section comprises an implant receiving section, such that the implant is disposed about at least a portion of the distal section.

Example 3. The system of any one of the preceding Examples, but particularly Example 1, wherein the tubular shaft is configured to be axially displaced, over a guidewire, in a lumen of a catheter to advance or retract the implant positioned on the tubular shaft.

Example 4. The system of any one of the preceding Examples, but particularly Example 2, further comprising a catheter, the catheter defining an inner diameter and configured to receive the tubular shaft therein, wherein the inner diameter of the catheter is sized relative to the outer diameter of the tubular shaft such that a sheath is not positionable in a lumen of the catheter when the tubular shaft, having the implant positioned thereon, is in the lumen of the catheter.

Example 5. The system of any one of the preceding Examples, but particularly Example 2, further comprising a guidewire, the guidewire being configured to be positioned in and translated in the lumen of the tubular shaft.

Example 6. The system of any one of the preceding Examples, but particularly Example 1, wherein the implant is a self-expanding stent.

Example 7. The system of any one of the preceding Examples, but particularly Example 2, wherein the distal section comprises the plurality of relief cuts, and the plurality of relief cuts comprises an interrupted spiral cut pattern.

Example 8. The system of any one of the preceding Examples, but particularly Example 1, wherein the proximal section comprises the plurality of relief cuts, and the plurality of relief cuts comprises a combination of an interrupted spiral cut pattern and a brick cut pattern.

Example 9. The system of any one of the preceding Examples, but particularly Example 8, wherein the interrupted spiral cut pattern and the brick cut pattern are in nonoverlapping regions of the proximal section.

Example 10. The system of any one of the preceding Examples, but particularly Example 1, wherein the outer diameter of the tubular shaft is sized relative to an inner diameter of a catheter configured to receive the tubular shaft therein, such that a sheath is not positionable in a lumen of the catheter when the tubular shaft is in the lumen of the catheter.

Example 11. The system of any one of the preceding Examples, but particularly Example 1, further comprising a backstop on a proximal end of the at least one aperture.

Example 12. The system of any one of the preceding Examples, but particularly Example 2, wherein the plurality of relief cuts is configured to prevent foreshortening or forelengthening of the tubular shaft.

Example 13. The system of any one of the preceding Examples, but particularly Example 2, wherein the proximal section, the implant coupling section, and the distal section are of a monolithic construction.

Example 14. The system of any one of the preceding Examples, but particularly Example 1, wherein the feature of the implant comprises a radiopaque marker radially extending from a proximal end of the implant.

Example 15. A method for delivering a vascular implant, the method comprising: advancing a guidewire and a catheter to a target site, wherein the guidewire is configured to be axially translated in the catheter; loading a tubular shaft over the guidewire and into a lumen of the catheter, wherein at least a portion of the tubular shaft has an implant disposed thereon; and axially translating the tubular shaft over the guidewire and through the lumen of the catheter.

Example 16. The method of any one of the preceding Examples, but particularly Example 16, further comprising deploying the implant at the target site by urging the tubular shaft out of a distal end of the lumen of the catheter.

Example 17. The method of any one of the preceding Examples, but particularly Example 16, wherein the loading comprises removing a sheath from the implant and the tubular shaft as the tubular shaft is loaded into the lumen of the catheter.

Example 18. The method of any one of the preceding Examples, but particularly Example 16, wherein axially translating the tubular shaft comprises advancing the tubular shaft through the lumen of the catheter and over the guidewire.

Example 19. The method of any one of the preceding Examples, but particularly Example 16, wherein axially translating the tubular shaft comprises retracting the tubular shaft in the lumen of the catheter and over the guidewire.

Example 20. The method of any one of the preceding Examples, but particularly Example 16, wherein the target site is an aorta fluidly connected to a ductus arteriosus.

Example 21. The method of any one of the preceding Examples, but particularly Example 16, wherein the target site is a pulmonary artery fluidly connected to a ductus arteriosus.

Example 22. The method of any one of the preceding Examples, but particularly Example 16, wherein the implant is a self-expanding stent.

Example 23. The method of any one of the preceding Examples, but particularly Example 16, further comprising axially translating the guidewire within a lumen of the tubular shaft.

Example 24. A system for delivering a vascular implant, the system comprising: a tubular shaft defining a lumen and comprising: a proximal section, an implant coupling section defining at least one aperture, and a plurality of relief cuts in the tubular shaft; an implant having a proximal end comprising at least one feature, wherein the implant is positioned on the tubular shaft, and the at least one feature is positioned in the at least one aperture of the tubular shaft; and a catheter defining a lumen having an inner diameter, wherein the inner diameter of the catheter is sized relative to an outer diameter of the tubular shaft such that a sheath is not positionable in the lumen of the catheter when the tubular shaft is in the lumen of the catheter, wherein the tubular shaft is configured to be axially displaced in the lumen of the catheter to advance or retract the implant positioned on the tubular shaft. Example 25. The system of any one of the preceding Examples, but particularly

Example 24, wherein the tubular shaft further comprised a distal section, wherein the implant coupling section is between the proximal section and the distal section, and wherein the distal section comprises an implant receiving section, such that the implant is disposed about at least a portion of the distal section.

Example 26. The system of any one of the preceding Examples, but particularly Example 24, further comprising a guidewire, wherein the tubular shaft is configured to be axially displaced, over the guidewire, in the lumen of the catheter to advance or retract the implant positioned on the tubular shaft.

Example 27. The system of any one of the preceding Examples, but particularly Example 24, wherein the implant is a self-expanding stent.

Example 28. The system of any one of the preceding Examples, but particularly Example 25, wherein the distal section comprises the plurality of relief cuts, and the plurality of relief cuts comprises an interrupted spiral cut pattern.

Example 29. The system of any one of the preceding Examples, but particularly Example 24, wherein the proximal section comprises the plurality of relief cuts, and the plurality of relief cuts comprises a combination of an interrupted spiral cut pattern and a brick cut pattern.

Example 30. The system of any one of the preceding Examples, but particularly Example 29, wherein the interrupted spiral cut pattern and the brick cut pattern are in nonoverlapping regions of the proximal section.

Example 31. The system of any one of the preceding Examples, but particularly Example 24, further comprising a backstop on a proximal end of the at least one aperture.

Example 32. The system of any one of the preceding Examples, but particularly Example 24, wherein the plurality of relief cuts is configured to prevent foreshortening or forelengthening of the tubular shaft.

Example 33. The system of any one of the preceding Examples, but particularly Example 25, wherein the proximal section, the implant coupling section, and the distal section are of a monolithic construction.

Example 34. The system of any one of the preceding Examples, but particularly Example 25, wherein the at least one feature of the implant comprises a radiopaque marker radially extending from the proximal end of the implant.

Example 35. A system for delivering an implant, the system comprising: a tubular shaft defining a lumen and comprising: a proximal section and a distal section, at least one aperture defined by at least the distal section, wherein the at least one aperture is configured to receive a radiopaque marker of an implant positioned on an outer diameter of the tubular shaft, and a plurality of relief cuts in the tubular shaft, the plurality of relief cuts having an interrupted pattern.

Example 36. The system of any one of the preceding embodiments, but particularly, Example 35, further comprising a catheter defining an inner diameter and configured to receive the tubular shaft therein, wherein the inner diameter of the catheter is sized relative to the outer diameter of the tubular shaft such that a sheath is not positionable in a lumen of the catheter when the tubular shaft is in the lumen of the catheter, wherein the tubular shaft is configured to be axially displaced in the lumen of the catheter to advance or retract the implant positioned on the tubular shaft.

Example 37. The system of any one of the preceding embodiments, but particularly, Example 35, further comprising a guidewire configured to be positioned in and translated in the lumen of the tubular shaft.

Example 38. A tubular shaft for delivering an implant, the tubular shaft comprising: a proximal section and a distal section; a lumen defined by the tubular shaft, wherein the lumen is configured to receive a guidewire therethrough; at least one aperture defined by at least the distal section, wherein the at least one aperture is configured to receive a radiopaque marker of the implant positioned on an outer diameter of the tubular shaft; and a plurality of relief cuts in the tubular shaft, the plurality of relief cuts having an interrupted pattern.

Example 39. The tubular shaft of any one of the preceding embodiments, but particularly, Example 38, wherein the outer diameter of the tubular shaft is sized relative to an inner diameter of a catheter configured to receive the tubular shaft therein, such that a sheath is not positionable in a lumen of the catheter when the tubular shaft is in the lumen of the catheter.

Example 40. The tubular shaft of any one of the preceding embodiments, but particularly, Example 39, wherein the tubular shaft is configured to be axially displaced in the lumen of the catheter to advance or retract the implant positioned on the tubular shaft.

Example 41. The tubular shaft of any one of the preceding Examples, but particularly Example 38, further comprising a bumper on a proximal end of the at least one aperture, the bumper configured to push the radiopaque marker of the implant when the tubular shaft is axially displaced in a lumen of a catheter.

Example 42. The tubular shaft of any one of the preceding Examples, but particularly Example 38, wherein the plurality of relief cuts forms a spine along a length of the tubular shaft.

Example 43. The tubular shaft of any one of the preceding Examples, but particularly Example 38, wherein the plurality of relief cuts is configured to prevent foreshortening or forelengthening of the tubular shaft.

Example 44. A tubular shaft for delivering an implant, the tubular shaft comprising: a proximal section and a distal section; a lumen defined by the tubular shaft, wherein the lumen is configured to receive a guidewire therethrough; at least one aperture defined by at least the distal section; and a plurality of relief cuts in the tubular shaft, the plurality of relief cuts having an interrupted pattern.

Example 45. The tubular shaft of any one of the preceding Examples, but particularly Example 44, wherein the outer diameter of the tubular shaft is sized relative to an inner diameter of a catheter configured to receive the tubular shaft therein, such that a sheath is not positionable in a lumen of the catheter when the tubular shaft is in the lumen of the catheter.

Example 46. The tubular shaft of any one of the preceding Examples, but particularly Example 44, wherein the tubular shaft is configured to be axially displaced in the lumen of the catheter to advance or retract the implant positioned on the tubular shaft.

Example 47. A method for delivering an implant, over a guidewire, within an empty catheter system, the method comprising: loading the implant over a tubular shaft; loading the tubular shaft over a guidewire; and advancing the tubular shaft through a lumen of a catheter, wherein the tubular shaft is configured to push the implant along a length of the lumen of the catheter to be deployed.

Example 48. A method for delivering an implant, over a guidewire, within an empty catheter system, the method comprising: loading the tubular shaft over a guidewire; and advancing or retracting the tubular shaft through a lumen of a catheter, wherein the tubular shaft is configured to push or pull, respectively, the implant through a length of the lumen of the catheter.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “aperture” may include, and is contemplated to include, a plurality of apertures. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for delivering a vascular implant, comprising:
a catheter defining a catheter lumen;
an implant;
a tubular shaft defining a lumen and configured to be positioned in the catheter lumen of the catheter without a sheath, the tubular shaft comprising:
a proximal section,
an implant coupling section bonded to the proximal section, wherein the implant coupling section defines at least one aperture, and wherein the at least one aperture is configured to receive a feature of the implant positioned on an outer diameter of the tubular shaft, and
a plurality of relief cuts in the tubular shaft to increase a flexibility of the tubular shaft; and
a guidewire configured to be axially displaced within the lumen of the tubular shaft,
wherein the guidewire and the catheter are configured to be advanced to a target site, and wherein the tubular shaft, with the implant disposed thereon, is configured to be loaded over the guidewire and into the catheter lumen of the catheter to be advanced to the target site through the catheter lumen.

2. The system of claim 1, wherein the tubular shaft further comprises a distal section, wherein the implant coupling section is between the proximal section and the distal section, and wherein the distal section comprises an implant receiving section, such that the implant is disposed about at least a portion of the distal section.

3. The system of claim 1, wherein the tubular shaft is configured to be axially displaced, over the guidewire, in the catheter lumen of the catheter to advance or retract the implant positioned on the tubular shaft.

4. The system of claim 2, wherein the catheter defines an inner diameter, wherein the inner diameter of the catheter is sized relative to the outer diameter of the tubular shaft such that the sheath is not positionable in the catheter lumen of the catheter when the tubular shaft, having the implant positioned thereon, is in the catheter lumen of the catheter.

5. The system of claim 1, wherein the implant is a self-expanding stent.

6. The system of claim 2, wherein the distal section comprises the plurality of relief cuts, and the plurality of relief cuts comprises an interrupted spiral cut pattern.

7. The system of claim 1, wherein the proximal section comprises the plurality of relief cuts, and the plurality of relief cuts comprises a combination of an interrupted spiral cut pattern and a brick cut pattern.

8. The system of claim 7, wherein the interrupted spiral cut pattern and the brick cut pattern are in nonoverlapping regions of the proximal section.

9. The system of claim 1, wherein the outer diameter of the tubular shaft is sized relative to an inner diameter of the catheter configured to receive the tubular shaft therein, such that the sheath is not positionable in the catheter lumen of the catheter when the tubular shaft is in the catheter lumen of the catheter.

10. The system of claim 1, further comprising a backstop on a proximal end of the at least one aperture.

11. The system of claim 2, wherein the plurality of relief cuts is configured to prevent foreshortening or forelengthening of the tubular shaft.

12. The system of claim 2, wherein the proximal section, the implant coupling section, and the distal section are of a monolithic construction.

13. The system of claim 1, wherein the feature of the implant comprises a radiopaque marker radially extending from a proximal end of the implant.

14. A system for delivering a vascular implant, comprising:
a tubular shaft defining a lumen and comprising:
a proximal section, and
an implant coupling section defining at least one aperture;
an implant having a proximal end comprising at least one feature, wherein the implant is positioned on the tubular shaft, and the at least one feature is positioned in the at least one aperture of the tubular shaft; and
a catheter defining a catheter lumen having an inner diameter, wherein the inner diameter of the catheter is sized relative to an outer diameter of the tubular shaft such that a sheath is not positionable in the catheter lumen of the catheter when the tubular shaft is in the catheter lumen of the catheter,
wherein the tubular shaft is configured to be axially displaced in the catheter lumen of the catheter to advance or retract the implant positioned on the tubular shaft.

15. The system of claim 14, wherein the tubular shaft further comprised a distal section, wherein the implant coupling section is between the proximal section and the distal section, and wherein the distal section comprises an implant receiving section, such that the implant is disposed about at least a portion of the distal section.

16. The system of claim 14, further comprising a guidewire, wherein the tubular shaft is configured to be axially displaced, over the guidewire, in the catheter lumen of the catheter to advance or retract the implant positioned on the tubular shaft.

17. The system of claim 15, wherein:
the distal section comprises a plurality of relief cuts;
the plurality of relief cuts comprises an interrupted spiral cut pattern; and
the plurality of relief cuts is configured to prevent foreshortening or forelengthening of the tubular shaft.

18. The system of claim 14, wherein:
the proximal section comprises a plurality of relief cuts;
the plurality of relief cuts comprises a combination of an interrupted spiral cut pattern and a brick cut pattern; and
the plurality of relief cuts is configured to prevent foreshortening or forelengthening of the tubular shaft.

19. The system of claim 17, wherein the interrupted spiral cut pattern and the brick cut pattern are in nonoverlapping regions of the proximal section.

20. The system of claim 14, further comprising a backstop on a proximal end of the at least one aperture.
